SEQUENCE LISTING (3 sequences)

SEQ ID NO: 1 - protein, 452 aa, Artificial Sequence
  heavy chain
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Artificial Sequence
  light chain
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

(19) United States
(12) Patent Application Publication
(10) Pub. No.: US 2019/0218302 A1
(43) Pub. Date: Jul. 18, 2019

(54) ANTI-CANCER COMBINATION TREATMENT

(71) Applicant: PROBIOCON GMBH, Pfeffingen (CH)

(72) Inventors: Andreas HERRMANN, Pfeffingen (CH); Bastian Julius SCHMIED, Tübingen (DE); Stefanie MAURER, Tübingen (DE); Helmut SALIH, Tübingen (DE)

(21) Appl. No.: 16/303,092

(22) PCT Filed: May 15, 2017
(86) PCT No.: PCT/EP2017/061598

§ 371 Date: Nov. 19, 2018

(30) Foreign Application Priority Data
May 19, 2016 (EP) 16170328.5

(51) Int. Cl. C07K 16/28; A61K 31/12; A61K 9/00; A61P 35/00

(52) U.S. Cl. CPC C07K 16/2875; A61K 31/12; A61K 45/06; A61P 35/00; A61K 9/0019

(57) ABSTRACT

A RANKL-inhibitor for use in the treatment of a patient suffering from chemotherapy resistant cancer, which patient has a diagnosis of cancer that is unresponsive to treatment with a first chemotherapeutic agent, wherein said cancer is not a solid tumor or metastasis in the bone, and said patient is administered a second chemotherapeutic agent in combination with said RANKL-inhibitor.

### TECHNICAL FIELD

The invention refers to a RANKL-specific inhibitor which recognizes and optionally neutralizes human receptor activator of nuclear factor kappa-B ligand (RANKL), for use in treating a cancer patient suffering from chemotherapy resistant cancer.

### BACKGROUND

A large field of research and development focuses on the treatment of cancer. Products under development range from kinase inhibitors, to angiogenesis inhibitors, monoclonal antibodies against tumor targets, apoptosis inducers, anti-tumor vaccination, and conventional chemotherapeutic agents against various tumor targets and with various cytotoxic effects. Prognosis of cancer patients is mainly determined by the risk of developing metastasis.

Bone metastases are a frequent complication of many cancers that result in severe disease burden and pain. Regulation of cancer cell migration and bone metastasis by RANK (receptor activator of NF-kB) ligand (RANKL) is described by Jones et al. (Nature 2006, 440:692-696). RANKL triggers migration of human epithelial cancer cells and melanoma cells that express the receptor RANK. RANK is expressed on a series of cancer cell lines and cancer cells in patients. In a mouse model of melanoma metastasis, in vivo neutralization of RANKL by osteoprotegerin results in complete protection from paralysis and a marked reduction in tumor burden in bones, but not in other organs. RANKL produced by bone microenvironment is considered a fertile soil for RANK-positive tumor cells.

Dougall et al. (BoneKEy Reports 2014, 3:519) describes RANKL an essential mediator of osteoclast function and survival, acting through its cognate receptor, RANK. Preclinical data have firmly established that blockade of tumor-induced osteoclastogenesis by RANKL inhibition would not only protect against bone destruction, but would also inhibit the progression of established bone metastases and delay the formation of de novo bone metastases in cancer models. In patients with bone metastases, skeletal complications are driven by increased osteoclastic activity and may result in pathological fractures, spinal cord compression and the need for radiotherapy to the bone or orthopedic surgery (collectively known as skeletal-related events (SREs)). Denosumab, a fully human monoclonal antibody against RANKL and RANKL-inhibitor, is described to prevent or delay SREs in patients with solid tumors that have metastasized to bone. In addition to its central role in tumor-induced osteolysis, bone destruction and skeletal tumor progression, there is emerging evidence for direct pro-metastatic effects of RANKL, independent of osteoclasts. For example, RANKL also stimulates metastasis via activity on RANK-expressing cancer cells, resulting in increased invasion and migration.

Tan et al. (Nature 2011, 470 (7335):548-553) describe that fibroblast-recruited, tumor infiltrating CD4+ T cells stimulate mammary cancer metastasis through RANKL-RANK signalling.

WO2012/038504 discloses a method of treating breast cancer using a RANKL-inhibitor. It is described that inhibition or inactivation of RANKL or its receptor RANK in breast cancer cells prevents progestin-induced proliferation. The RANKL-inhibitor may be used in combination with a radiation- or chemotherapy.

Sharma and Singh (Journal of Carcinogenesis. 2011; 10:36) review the role of RANK-RANKL signaling in breast tumors and in mediating chemotherapy resistance in breast cancer cells. Studies propose that there is a loss of RANKL in breast tumors as they become more aggressive, but the one that retain RANKL are of higher histological grade.

Denosumab was approved by the U.S. Food and Drug Administration for use in postmenopausal women with risk of osteoporosis under the trade name Prolia, and as Xgeva, for the prevention of SREs in patients with bone metastases from solid tumors. Clinical trials were investigating Denosumab, among others, in giant cell tumors, multiple myeloma with bone metastases, and hypercalcemia of malignancy.

Therapies targeting RANK/RANKL e.g., involve RANKL-specific binders, among them Denosumab, recombinant RANK-Fc (Schmiedel et al. 2013, Cancer Res. 73(2):683-94), or RANKL-nanobodies (WO2008142164A2). RANKL-binding peptides are described to inhibit bone resorption and/or osteoclast activity (WO2012163887A1).

The effect of Denosumab on bone metastasis in patients with advanced solid tumors is described in a series of documents e.g., Rolfo Christian et al. (Expert Opinion on Biological Therapy, vol. 14, no. 1, 2014, pp 15-26), Scagliotti Giorgio Vittorio et al. (Journal of Thoracic Oncology, vol. 7, no. 12, 2012, pp 1823-1829), Morikawa K. et al. (Database Embase, Elsevier Science publishers, Amsterdam, XP002736136; and Japanese Journal of Lung Cancer, vol. 52, no. 7, 2012, pp 1035-1040), Takeshi Yuasa et al. (Oncotargets and Therapy, vol. 5, 2012, pp 221-229), Hilbe Wolfgang et al. (Magazine of European Medical Oncology, AT, vol. 6, no. 2, 2013, pp 75-82), Lászkó Kopper (Pathology & Oncology Research, vol. 18, no. 4, 2012, pp 743-747), Sonya J. Snedecor et al., (Clinical Therapeutics, vol. 34, no. 6, 2012, pp 1334-1349), Sarah Payton (Nature Reviews Urology, vol. 9, no. 1, 2011, pp 1-1), WO 2013/176469 and DATABASE WPI, Thomson Scientific, London, GB, XP002736138; US 2012/114665 A1, and WO 01/08699 A1).

Morikawa et al (Japanese Journal of Lung Cancer, vol. 52, no. 7, 2012, pp 1035-1040) describe the potential of Denosumab as a therapeutic alternative for bone metastasis if chemotherapy is ineffective.

Cathomas et al. (Oncology 2015, 88(4): 257-260) describe treatment of unresectable osteosarcoma by the RANKL blockade with Denosumab in combination with sorafenib.

Miller et al. (Molecular Cancer Therapeutics 2008, 7(7): 2160-2169) describe the combination of the RANKL inhibitor osteoprotegerin-Fc (OPG-Fc) with docetaxel in a murine model of prostate cancer bone metastasis.

Rousseau et al. (Journal of Bone and Mineral Research 2011, 26(10): 2452-2462) describe siRNAs targeting Rankl to enhance the chemotherapeutic response in osteosarcoma models.

Bago-Horvath et al. (Pathology 2014, 46(5): 411-415) describe the impact of RANK signaling on chemotherapy response in osteosarcoma.

Chemotherapy resistance is a major obstacle for the success of cancer therapy, in particular in cancers other than osteosarcoma, skeletal cancer or bone metastasis.

There is an urgent need to develop new therapies to overcome chemoresistance of tumors or to increase the sensibility of tumors to antineoplastic or chemotherapeutic drugs.

### SUMMARY OF THE INVENTION

It is the object of the invention to provide for an improved treatment of chemotherapy resistance in cancer patients, and respective chemotherapy combination therapies.

The object is solved by the subject matter of the invention.

The invention provides for a RANKL-inhibitor for use in the treatment of a patient suffering from chemotherapy resistant cancer, which patient has a diagnosis of a cancer that is unresponsive to treatment with a first chemotherapeutic agent, wherein said cancer is not a solid tumor in the bone, or metastasis in the bone, and wherein said patient is administered a second chemotherapeutic agent in combination with said RANKL-inhibitor.

Specifically, the cancer is a solid tumor or a disease associated with a solid tumor, which is localized in any part of the human body except the bone. Specifically, osteosarcoma or solid tumors of skeletal origin and/or located in the bone are excluded from treatment as described herein.

Specifically, the cancer is a metastasis or a disease associated with metastases, which is/are localized in any part of the human body except the bone. Specifically, bone metastases or metastases located in the bone are excluded from treatment as described herein.

Specifically, the treatment is not targeting any of bone cancer, bone metastasis, osteosarcoma or osteogenic sarcoma, or patients suffering from any of the foregoing.

Specifically, the cancer patient has a diagnosis or suffers from carcinoma. Carcinoma is generally understood as a type of cancer that develops from epithelial cells and includes e.g. cancer of the skin, head, neck, lungs, breast, pancreas, colon, prostate, ovary, liver, kidney, intestine, and other organs and glands. Specifically, the carcinoma is any of adenocarcinoma, squamous cell carcinoma, adenosquamous carcinoma, anaplastic carcinoma, large cell carcinoma, or small cell carcinoma.

Specifically, the cancer patient has a diagnosis or suffers from blood cancer, which is generally understood as any kind of hematological malignancy, Specifically, the blood cancer is any of leukemia or lymphoma (or cancers of lymphocytes). Specifically, the leukemia is any of acute lymphoblastic leukemia (ALL), acute myelogenous leukemia (AML), chronic lymphocytic leukemia (CLL), small lymphocytic lymphoma (SLL), chronic myelogenous leukemia (CML), or acute monocytic leukemia (AMoL. Specifically, the lymphoma is any of Hodgkin's lymphoma (all subtypes), or Non-Hodgkin's lymphoma (all subtypes).

Specifically, the cancer is a RANK positive or RANK expressing cancer.

Specifically, the cancer is RANK negative cancer. RANKL may be reverse signaled in cancer cells, e.g. by RANK positive stroma, or interaction between cancer cells and the stromal cells that are present in the tumor microenvironment.

Specifically, said patient has failed treatment with said first chemotherapeutic agent and/or the unresponsiveness of the cancer cells was determined in an ex vivo assay, and/or which is otherwise known to be chemotherapy resistant. In particular, said cancer is resistant to said first chemotherapeutic agent without the combination with said RANKL-inhibitor, as determined by in an ex vivo assay or by previous or historic experimental data.

Such ex vivo assay typically employs cancer cells of cell lines, biological samples or isolates from the cancer patient, and means for determining the sensitivity or resistance of such cancer cells in the presence of chemotherapeutic agents, e.g., cytotoxic small molecule (organic) drugs, with or without the RANKL-inhibitor.

Specifically, said cancer is known to be chemotherapy resistant with respect said first chemotherapeutic agent, or at least known to be substantially chemotherapy resistant, such that the chemotherapeutic agent is not administered to said patient without combination with the RANKL-inhibitor as described herein. Specifically, said cancer is sensitized to chemotherapy with respect said first chemotherapeutic agent, such that the administered dose or amount of said first chemotherapeutic agent administered to said patient is reduced when combined with the RANKL-inhibitor as described herein.

Specifically, said patient has suffered disease progression after said treatment with said first chemotherapeutic agent including a cytotoxic chemotherapy regime, preferably a minimum of two or three courses of one prior cytotoxic chemotherapy regime for said cancer. The cytotoxic chemotherapy regime typically involves one or more cytotoxic agents. An example is the FEC (fluorouracil, epirubicin, cyclophasmide) regime commonly used in the treatment of metastatic breast cancer. In this regime fluorouracil, epirubicin, cyclophasmide are applied every 21 days, with 6 to 8 cycles in total. Another example is the FOLFIRI (Folinic acid, fluorouracil, irinotecan) regime used as chemotherapy for the treatment of metastatic colorectal cancer. This regime is given 2-weekly in multiple cycles.

Specifically, said first chemotherapeutic agent is a cytostatic, cytotoxic or any other anti-neoplastic compound used for treating a cancer disease.

Exemplary chemotherapeutic agents are selected from the group consisting of 5-fluorouracil, capecitabine, carboplatin, cisplatin, gemcitabine, irinotecan, oxaliplatin, topotecan, 6-thioguanine, mitomycin-c, paclitaxel, docetaxel, dacarbacine, etoposide, pemetrexed, cyclophosphamide, doxorubicin, vincristine, amrubicin, temozolomide, a platinum agent, and a combination of any of the foregoing.

Specifically, said second chemotherapeutic agent is administered at a substantially or significantly reduced dose as compared to standard chemotherapy without combination with said RANKL-inhibitor, e.g., at a dose which is reduced by at least any of 10%, 20%, 30%, 40%, 50%, 60%, 70%, 80%, or 90%; or by substantially reducing the frequency of chemotherapy or the number of treatment cycles.

Specifically, said first chemotherapeutic agent is the same or differs from said second chemotherapeutic agent.

According to particular examples, the patient suffers from breast cancer, which is unresponsive to treatment with paclitaxel, and treated with paclitaxel in combination with the RANKL-inhibitor. In particular, the breast cancer patient undergoes a first treatment employing paclitaxel (as a first chemotherapeutic agent), which is followed by a second treatment using paclitaxel (as a second chemotherapeutic agent) concomitantly with the RANKL-inhibitor, e.g., an antibody targeting RANKL such as Denosumab.

According to a further particular example, the patient suffers from acute myeloid leukemia (AML), which is unresponsive to treatment with doxorubicin, and treated with doxorubicinin in combination with the RANKL-inhibitor. In particular, the AML patient undergoes a first treatment employing doxorubicin (as a first chemotherapeutic agent), which is followed by a second treatment using doxorubicin (as a second chemotherapeutic agent) concomitantly with the RANKL-inhibitor, e.g., an antibody targeting RANKL such as Denosumab.

According to a specific aspect, the said cancer is a solid tumor selected from the group consisting of epithelial tumors, mesenchymal tumors, tumors of endodermal, mesodermal and/or ectodermal origin, or a blood-borne cancer, such as leukemia or lymphoma. Specifically, said patient suffers from any of such solid tumors or blood cancer and is treated with the combination therapy as described herein, including a chemotherapeutic agent for sensitizing against said chemotherapeutic agent by combining with said RANKL-inhibitor. Specifically, said RANKL-inhibitor is administered in an effective amount to sensitize said cancer for an improved chemotherapy.

Specifically, the patient suffers from breast cancer, pancreatic cancer, gastric cancer, esophageal cancer, renal cell carcinoma, lung carcinoma, colon/rectal/colorectal cancer, melanoma, ovary cancer, liver cancer, kidney cancer, intestine cancer, prostate cancer, head and neck cancer, lymphoma or leukemia. Specifically, the patient is suffering from a primary solid tumor or cancer of the blood or the lymphatic system.

Specifically, the cancer is characterized by RANK-positive cancer or metastasis cells. The cancer cells can be RANK-positive tumor-forming cancer cells or tumor cells, in particular solid tumor cells, or RANK positive cancer cells which involve the blood and blood-forming organs, e.g., leukemia. Specifically, the cancer is characterized by the presence of premetastatic lesions which are identified by determining activated platelet-cancer cell aggregates, or circulating premetastatic cell clusters forming niches, thereby promoting cancer metastasis or increasing the risk of developing metastasis, e.g., in distant organs or bone metastasis.

Specifically, the cancer patient is at risk of or suffering from minimal residual disease and/or recurrence of metastatic disease, optionally wherein the patient has a detectable level of circulating tumor cells in a blood sample, e.g., as determined by the number of disseminated tumor cells in whole blood or a blood fraction thereof, or by specific tumor cell marker. A detectable number of tumor cells is e.g., less than 10, or less than 5, or 4, 3, 2, or 1 circulating tumor cells in a sample of whole blood of at least 5 mL, or 7.5 mL, or 10 mL.

According to a specific aspect, the RANKL-inhibitor is selected from the group consisting of antibodies, antibody fragments, receptor-fusion proteins, such as RANK-Fc fusion proteins, peptides, such as inactivated forms of osteoprotegerin, or fragments thereof, small molecules, such as RANK-specific organic small molecules, or aptamers. Exemplary small molecules are small molecule inhibitors of RANKL and TNF, such as described in Caste E, et al. (Ann Rheum Dis 2015; 74:220-226). Specific examples are derivatives of butanediol biphenylcarboxylic acid ester, which are capable of inhibiting RANKL-induced phosphorylation of IκB and extracellular signal-regulated kinase (ERK). For example, compounds where the ester bond is replaced by a ketone may be used, such as ABD328 and ABD345 characterized by the following formula:

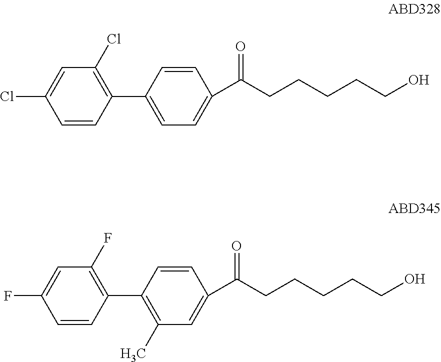 [SMILES: O=C(CCCCCO)c1ccc(-c2ccc(Cl)cc2Cl)cc1 ; Cc1cc(C(=O)CCCCCO)ccc1-c1ccc(F)cc1F]

Specifically, the RANKL-inhibitor is any of a human or humanized antibody, an antigen-binding fragment thereof, or a RANKL receptor-Fc fusion protein, such as a RANK-Fc fusion protein or a LGR4-Fc fusion protein, a molecule comprising the extracellular domain of a RANKL receptor, such as RANK or LGR4, or a small molecule inhibitor, such as RANK-specific organic small molecules.

According to a specific example, the RANKL-inhibitor is a human or humanized antibody, such as Denosumab, a generic or biosimilar version, or a functional variant thereof, or an antigen-binding fragment of any of the foregoing, or a RANK-Fc fusion protein. Denosumab (Amgen, Thousand Oaks, Calif., USA) is a fully human IgG2 monoclonal antibody specific to RANKL, which is described to suppress bone resorption markers in patients with a variety of metastatic tumors and is being investigated in multiple clinical trials for the prevention and treatment of bone metastases. Chemically, it consists of 2 heavy and 2 light chains. Each light chain consists of 215 amino acids. Each heavy chain consists of 448 amino acids with 4 intramolecular disulfides. The heavy chain amino acid sequence is identified by SEQ ID 1; the light chain amino acid sequence is identified by SEQ ID 2.

Specifically, the agent comprises an Fc antibody fragment, such as a human IgG1 Fc, which is engineered to reduce Fc effector function (e.g., which does not significantly bind to the FcgammaRIIIa, or CD16), and therefore does not exhibit significant antibody-dependent cellular cytotoxicity (ADCC). Exemplary Fc fragments which comprise point mutations to reduce Fc effector function are characterized by at least one of the following mutations: E233P, L234V, L235A, deltaG236, A327G, A330S, wherein nomenclature is according to the EU index of Kabat.

Alternatively, the agent comprises an Fc antibody fragment, such as a human IgG1 Fc, with Fc effector function (e.g., binding to the FcgammaRIIIa, or CD16), such as ADCC. Such agent would have the additional advantage of cell-mediated immune defense whereby an effector cell of the immune system actively destroys the target cell, which is the platelet and/or the cancer cell, preferably the cancer-platelet aggregate.

The RANKL-inhibitor may specifically recognize and neutralize soluble RANKL (sRANKL) and/or or cell membrane-bound RANKL (mRANKL), and/or platelet-bound RANKL (pRANKL).

Specifically, the RANKL-inhibitor is recognizing the RANKL polypeptide, which may comprise the full amino acid sequence of human RANKL (SEQ ID 3), or an epitope in the extracellular portion of a membrane-bound RANKL, e.g., AA 69-AA 317 of SEQ ID 3), in particular competing with the binding of RANK to RANKL, and thereby substantially inhibiting the RANK-RANKL signaling in cancer cells. Specifically, the RANKL-inhibitor binds to RANKL, thereby inhibiting RANKL from activating its receptor on cancer cells, e.g., on primary tumor cells, disseminating or metastasizing tumor cells.

Specifically, the RANKL-inhibitor is recognizing and capable of binding to RANKL monomer, or multimer, such as a multimer of RANKL molecules interacting on the surface of aggregating cells. Such multimer may be a dimer, or trimer, or higher multimer, preferably forming a complex with membrane-bound RANKL and/or soluble RANKL. For example, binding may occur, e.g., on the surface of the platelet, in the microenvironment between a platelet and a cancer cell, or in the circulation.

According to a specific aspect, the RANKL-inhibitor is administered to the patient in a therapeutically effective amount by systemic administration, preferably by intravenous infusion or bolus injection.

Prior art therapy with Denosumab would typically involve subcutaneous treatment. The present invention would target activated circulating platelets expressing RANKL, or circulating platelet-cancer cell aggregates. Therefore, the intravenous route is specifically preferred.

Preferred RANKL-inhibitor doses are, e.g., ranging from 0.5 to 1000 mg, preferably 1-400 mg. If administered subcutaneously, the preferred dosage is ranging from 0.5 to 400 mg.

Specifically, the respective amounts of the RANKL-inhibitor and said second chemotherapeutic agent are effective to increase the cancer sensitivity and/or treat cancer cell resistance to the chemotherapeutic agent.

According to a specific embodiment, the RANKL-inhibitor and said second chemotherapeutic agent are administered concomitantly. Concomitant treatment is specifically given in a treatment regimen, wherein each of the RANKL-inhibitor and chemotherapeutic agent are administered within a short timeframe. Concomitant administration typically is by intraveneous treatment with the RANKL-inhibitor, and oral or parenteral administration of the chemotherapeutic agent, each within the same treatment course, e.g., within 12 or 24 hours, or within 1 or more several days, such as within 1, 2, 3, 4, 5, 6, or 7 days.

According to another specific embodiment, the RANKL-inhibitor is administered prior to, concurrently, and/or following said second chemotherapeutic agent. The combined administration may e.g., involve one or more interchanging administration of both active agents, and in particular an i.v. treatment course administering the RANKL-inhibitor within 12 hours, and another short-term or maintenance treatment (simultaneous, before or after) with the chemotherapeutic agent, e.g., within 1-3 days (short-term) or for more than 4, 5, or 6 days (long-term, maintenance). Combination administration may employ appropriate intervals.

According to a specific aspect, the RANKL-inhibitor is administered to the patient in combination with an adjuvant or neoadjuvant combination therapy, preferably further combining with a kinase inhibitor therapy and/or immunotherapy. Such combination therapy would specifically target the cancer cell, e.g., any tumor associated antigen, such as selected from the group consisting of epithelial cancer cell marker, soluble factors, or anti-angiogenic therapy.

According to a specific aspect, the treatment is combined with surgical intervention to remove at least part of a tumor, and/or combined with radiotherapy, and the RANKL-inhibitor is administered for neoadjuvant or adjuvant therapy. Accordingly, the patient specifically is preparing for or undergoing surgical intervention and/or radiotherapy, or has been treated by a surgical intervention and/or radiotherapy, and is further treated with the RANKL-inhibitor according to the invention before or after surgery. According to specific examples, such treatment may start between 1 to 30 days before surgery, or during surgery, or within 1 to 30 days after surgery, and the RANKL-inhibitor may be administered for a continued period, e.g., for 1 to 12 months, or even longer, wherein the RANKL-inhibitor is administered in regular intervals. Surgical interventions are e.g., therapeutic removal of tumor mass, or biopsy. Surgery is considered a specific risk factor of disseminating tumor cells into the blood stream, thereby provoking platelet-tumor cell aggregate formation. Likewise, radiation therapy can trigger the haematogeneous spread of tumor cells. Therefore, the method of the invention specifically is indicated in combination with surgery and/or radiotherapy which potentially disseminates solid tumor cells.

The invention further provides for a RANKL-inhibitor for use in the treatment of a cancer patient in combination with an anti-cancer treatment, wherein the anti-cancer treatment is administered at a substantially reduced dose as compared to standard treatment without combination with said RANKL-inhibitor. The standard dose may be any of a dose of a pharmaceutical compound, like a chemotherapeutic agent or an immunotherapeutic preparation or drug, and/or a dose of a physical treatment, such as irradiation. Specifically, said cancer is any of the cancer or disease associated with cancer as described herein, except a solid tumor in the bone, or metastasis in the bone.

The invention further provides for a RANKL-inhibitor for use in the treatment of a cancer patient in combination with a chemotherapeutic agent, wherein the chemotherapeutic agent is administered at a substantially reduced dose as compared to standard chemotherapy without combination with said RANKL-inhibitor. Specifically, said chemotherapeutic agent is administered at a substantially reduced dose as compared to standard chemotherapy without combination with said RANKL-inhibitor at a dose which is reduced by at least any of 10%, 20%, 30%, 40%, 50%, 60%, 70%, 80%, or 90%; or by substantially reducing the frequency of chemotherapy or the number of treatment cycles. Specifically, said cancer is any of the cancer or disease associated with cancer as described herein, except a solid tumor in the bone, or metastasis in the bone.

Likewise, the RANKL-inhibitor may be used in combination with any other anti-cancer treatment where there is a need to lower the (effective) dose to an acceptable dose, e.g., irradiation treatment and/or immunotherapy. Such dose reduction is specifically indicated, if a patient suffers from side effects of such anti-cancer treatment, such that it causes treatment arrest.

The invention further provides for a method of screening patients suffering from a cancer and who might benefit from an therapy for sensitizing the cancer cells to chemotherapy, said method comprising the step of measuring the level of expression and/or activity of RANK or RANKL in a biological sample of said patient, wherein patients who show an expression or an overexpression of RANK or RANKL are selected as a candidate for said sensitizing therapy. Specifically, said cancer is any of the cancer or disease associated with cancer as described herein, except a solid tumor in the bone, or metastasis in the bone.

The sensitizing therapy specifically includes administration of the RANKL-inhibitor in combination with the chemotherapeutic agent as described herein. Therefore, a specific aspect refers to the RANKL-inhibitor for use in treating the cancer patient, wherein the patient is first identified as a candidate for chemosensitizing therapy, followed by the therapy administering an effective amount of the RANKL-inhibitor in combination with a chemotherapeutic agent. The chemosensitizing therapy is optionally provided as adjuvant or a neoadjuvant therapy.

Specifically, the invention provides for the RANKL-inhibitor for use as described herein, wherein a patient is treated who has been identified as candidate for such treatment as described herein, in particular a chemosensitizing treatment with the RANKL-inhibitor.

Specifically, a method is provided wherein in a first step the cancer patient is identified as a candidate for chemosensitizing therapy by measuring the overexpression of RANK or RANKL in a biological sample of the patient; and in a second step the patient is treated with an effective amount of the RANKL-inhibitor to render the cancer responsive to a chemotherapeutic agent. Specifically, such treatment with the RANKL-inhibitor is in combination with a chemotherapeutic agent thereby resulting in an effective response to such treatment.

The method of screening patients may specifically further employ a prognostic assay based on the method of predicting the metastatic potential of a tumor or cancer, which can be used to determine whether a patient is suitably treated with a chemotherapeutic agent, to treat cancer or other disorders associated with cancer such as metastatic disease. For example, such assay can be used to determine whether a patient shall be administered with a chemotherapeutic agent.

For example, the level of expression and/or activity of RANK or RANKL is determined in cancer cells within the sample or isolated from the sample and compared to a reference value, e.g., such as obtained upon expression of the RANK or RANKL in predetermined chemosensitive cancer cells (serving as reference cells).

The level of RANKL expression is e.g., determined by the expression of a nucleotide sequence indicative of RANKL expression or encoding the RANKL (or a complementary sequence), or by the RANKL polypeptide, or a fragment thereof. The level may be determined qualitatively, but also semi-quantitatively, or quantitatively.

The reference value may be derived from a positive or negative control, or both. The positive control is e.g., representing the level of RANKL expression on chemoresistant cancer cells, e.g., obtained from a cancer patient suffering from such chemoresistance. The negative control is e.g., representing the RANKL expression level of chemosensitive cancer cells or obtained from tissue of a healthy control subject.

Specifically, said biological sample is selected from the group consisting of tissue samples obtained from a biopsy procedure or from a surgical procedure to remove a tumor mass, blood, which may comprise tumor derived material such as tumor cells or tumor relapsed proteins and/or nucleic acids.

Specifically, said method further comprises comparing the level of expression of RANK or RANKL to a threshold value, and wherein said threshold value is the mean level of expression of a population of patients who are healthy or who recovered from a cancer.

According to a specific further aspect, the invention provides for a method of increasing efficacy of a chemotherapeutic treatment in a cancer patient, comprising administering to the patient an effective amount of a RANKL-inhibitor as a second line treatment following said chemotherapeutic treatment. Specifically, said cancer is any of the cancer or disease associated with cancer as described herein, except a solid tumor in the bone, or metastasis in the bone.

According to a specific further aspect, the invention provides for a method of delaying and/or preventing development of cancer resistant to a chemotherapeutic treatment in a cancer patient, comprising administering to the patient an effective amount of a RANKL-inhibitor as a second line treatment following said chemotherapeutic treatment. Specifically, said cancer is any of the cancer or disease associated with cancer as described herein, except a solid tumor in the bone, or metastasis in the bone.

According to a specific further aspect, the invention provides for a method of increasing sensitivity to a chemotherapeutic treatment in a cancer patient, administering to the patient an effective amount of a RANKL-inhibitor as a second line treatment following said chemotherapeutic treatment. Specifically, said cancer is any of the cancer or disease associated with cancer as described herein, except a solid tumor in the bone, or metastasis in the bone.

Specifically, any of the treatment methods described herein comprises a chemotherapeutic treatment including the administration of a chemotherapeutic agent, and further comprises administering to the patient an effective amount of said chemotherapeutic agent.

### FIGURES

FIG. 1: RANKL mediates resistance of malignant cells to chemotherapeutic treatment. Blood of a patient suffering from acute myeloid leukemia (AML) containing 94% blasts (leukemic cells) was obtained at time of diagnosis prior to treatment and peripheral blood mononuclear cells (PBMC) were isolated by density gradient centrifugation. PBMC were cultured in the presence or absence of doxorubicin (dox, 1 μM) and/or soluble RANKL (sRANKL, 500 ng/ml) as indicated. Survival and cell death were analyzed after 48 h by flow cytometry (7AAD and Annexin-V-PE staining) and are displayed as contour plots (upper panel) and percent of analyzed cells (lower panel). The presented results demonstrate that RANKL protects the malignant cells from the effects of chemotherapeutic treatment, i.e. mediates treatment resistance.

FIG. 2: Blockade of RANKL by Denosumab overcomes resistance to chemotherapeutic treatment. MCF10A breast tumor cells were cultured for 48 h in the presence or absence of sRANKL (100 ng/ml) and/or Denosumab (20 μg/ml) as indicated. After the first 24 h of culture, paclitaxel (10 nM) was added where indicated. Then the percentage of living and dead cells was determined using flow cytometry with 7AAD. Results are displayed as contour plots (upper panels) and percentage of living (7AAD negative) and dead (7AAD positive) cells (lower panel). The presented results demonstrate RANKL protects the malignant cells from the effects of chemotherapeutic treatment and neutralization of RANKL by Denosumab serves to restore sensitivity to treatment.

FIG. 3: Sequences

Denosumab heavy and light chain amino acid sequences:

SEQ ID 1: heavy chain

SEQ ID 2: light chain

Human RANKL amino acid sequence (GenBank: AAB86811.1):

SEQ ID 3: full-length sequence

FIG. 4: Increased susceptibility of malignant cells to chemotherapy upon neutralisation of pRANKL by Denosumab

A2780 ovarian cancer cells (left) and MCF10A breast cancer cells (right) were employed as model to study the influence of RANKL and its neutralization on chemotherapy resistance. To this end, 30,000 tumor cells were cultured in the presence or absence of platelets and Denosumab for 96 h. The indicated concentrations of doxorubicin (left) or paclitaxel (right) were added for the last 24 h. Then cell viability was determined by analysis of ATP content of each sample (CellTiter-Glo assay, Promega). Values obtained with untreated tumor cells were set to 1.

### DETAILED DESCRIPTION OF THE INVENTION

The term “adjuvant” as used herein shall refer to the treatment of cancer during or after a surgical intervention and/or radiotherapy, e.g., for improved therapy.

The term “neoadjuvant” as used herein shall refer to the treatment of cancer prior to a surgical intervention and/or radiotherapy, e.g., for improved therapy.

The terms “chemotherapy resistant” and “chemoresistant” are herein used interchangeably and shall refer to a tumor or cancer cell that shows little or no significant detectable therapeutic response to an agent used in chemotherapy.

A “chemotherapy resistant cancer” is herein understood as a cancer in a patient where the proliferation of cancer cells cannot be prevented or inhibited by means of a chemotherapeutic agent or a combination of chemotherapeutic agents usually used to treat such cancer, at an acceptable dose to the patient. Tumors can be intrinsically resistant prior to chemotherapy, or resistance may be acquired during treatment by tumors that are initially sensitive to chemotherapy. Drug resistance can be defined by the amount of anti-cancer drug that is required to produce a given level of cell death. Clinical drug resistance can be defined either as the lack of reduction in the size of the tumor following chemotherapy or as the occurrence of clinical relapse after an initial positive response to the treatment.

The terms “chemotherapy sensitive” or “chemosensitive” are herein used interchangeably and shall refer to a tumor or cancer cell that shows a detectable therapeutic response to an agent used in chemotherapy.

The term “chemotherapy sensitive” is herein understood as a cancer in a patient which is responsive to the effects of a chemotherapeutic agent, i.e. where the proliferation of cancer cells can be prevented by means of said chemotherapeutic agent at an acceptable dose to the patient.

By “acceptable dose to the patient”, it is meant a dose which does not cause treatment arrest due to side effects.

The term “chemotherapy” as used herein shall refer to the treatment of cancer with chemical compounds that have a specific toxic effect upon the cancer, e.g., by interfering with cell reproduction.

The term “chemotherapeutic agent” is herein understood as an active substance, active ingredient, or the respective drug or combination of drugs, and is an antineoplastic agent or antitumor agent that has the effect of inhibiting the maturation, growth or proliferation, or inducing the killing, of a tumor or cancer cell. The chemotherapeutic agent may inhibit or reverse the development or progression of a tumor or cancer, such as for example, a solid tumor or blood cancer.

Chemotherapeutic agents are commonly understood as cytotoxic agents and classified according to their mode of action. The various classes of antineoplastic agents include in particular DNA-damaging agents, anti-tumor antibiotics, antimetabolites, antimitotics and miscellaneous antineoplastic agents.

DNA-damaging agents include alkylating agents and topoisomerase inhibitors.

Alkylating agents have the ability to alkylate many nucleophilic functional groups under conditions present in cells. They impair cell function by forming covalent bonds with the amino, carboxyl, sulfhydryl, and phosphate groups in biologically relevant molecules.

Antitumor antibiotics include anthracyclines, such as e.g., doxorubicin, and other anti-tumor antibiotics.

Antimetabolites are artificial compounds similar in structure to naturally-occurring compounds that are required for the viability and division of a cell. The efficacy of the most important anti-metabolites against a range of tumor cells is based on the inhibition of purine or pyrimidine nucleoside synthesis pathway required for DNA synthesis.

Antimitotics include e.g., taxanes, such as paclitaxel, docetaxel, mitoxantrone, vinblastine and estramustine, interfering with microtubules thereby inhibiting or stopping mitosis, and vinca alkaloids which bind to specific sites on tubulin, inhibiting the assembly of tubulin into microtubules.

The term “chemotherapy” as used herein shall refer to administration of at least one chemotherapeutic agent to a patient suffering from a tumor or cancer disease.

The terms “response” or “responsive” with respect to a cancer or tumor, is herein understood as the effect of treatment with a chemotherapeutic agent which is an improvement in at least one relevant clinical parameter as compared to an untreated patient diagnosed with such cancer or tumor, or as compared to the clinical parameters of the same subject prior to said treatment.

The term “non-response” or “non-responsive” to treatment with a chemotherapeutic agent as used herein shall refer to a treated cancer patient not experiencing an improvement in at least one of the clinical parameters. This term also encompasses a poor response to therapy which indicates a very low level of response which is not clinically significant or sufficient. For example, low responsiveness to a chemotherapeutic agent treatment may be reflected by less tumour shrinkage (e.g., compared to a positive responder, or the positive readout of a respective clinical study), or poor survival.

The responsiveness or non-responsiveness can be determined upon such treatment of the patient with the chemotherapeutic agent and assessing the respective clinical parameter, such as overall survival, progression-free survival, grade or stage of disease.

Responsiveness or non-responsiveness may as well be predicted by an ex vivo assay using cells of the respective cancer or tumor, e.g., cells obtained from a sample of the patient's cancer or tumor. Parameters indicating the respective response to a chemotherapeutic agent may be obtained from a vital property or function of the cell, including e.g., the phosphorylation status. Alternatively, the cells can be visually examined for determining and differentiating live, damaged, or dead cells.

According to a specific example, the patient's response to chemotherapy or a respective chemoresistance is conveniently determined using clonal cell lines, or a patient-derived xenograft (PDX) model employing a sample of a patient's cancerous tissue such as from a patient's primary tumor, implanted into an immunodeficient mouse. Such PDX model would allow examining efficacy of a therapy, but also basic tenets of cancer biology, e.g., oncogene expression profiles, in response to treatment.

Typically, chemotherapy for specific cancer types is given on the basis of empirical information from clinical trials, however, the heterogeneity of chemosensitivity between patients is a well-known phenomenon. Therefore, tumor chemosensitivity assays are provided which have been correlated prospectively with patient response (e.g., Cree et al. 1996 Anticancer Drugs; 7(6):630-5). Several ex vivo assays are available in the art which are designed to predict the sensitivity and resistance of a given patient's solid tumor to a variety of chemotherapy agents. For example, a portion of a patient's solid tumor, such as a biopsy, is mechanically disaggregated and established in primary culture where malignant epithelial cells migrate out of tumor explants to form a monolayer. Cultures can be verified as epithelial and exposed to increasing doses of selected chemotherapeutic agents. The number of live cells remaining post-treatment can be enumerated microscopically e.g., using automated cell-counting software. The resultant cell counts in treated wells can be compared with those in untreated control wells to generate a dose-response curve for each chemotherapeutic agent tested on a given patient specimen. Features of each dose-response curve can be used to score a tumor's response to each ex vivo treatment as “responsive,” “intermediate response,” or “non-responsive.” Collectively, such scores can be used to assist an oncologist in making treatment decisions.

In case a previous or first treatment with a chemotherapeutic agent fails to result in a positive response, a further line of treatment, herein understood as “second-line treatment” may be indicated, either with a different chemotherapeutic agent or with a combination treatment, such as described herein. A second-line treatment is herein specifically understood as a treatment that is given when initial or previous treatment (e.g., the first-line therapy) does not work, or stops working. The term “second-line” specifically includes any further treatment line following a first or previous treatment. Thus, the term includes any second-line treatment following a first-line treatment, and any further one or more treatment lines which may follow the second-line treatment.

First-line therapy or further lines of therapy are typically chosen over other options usually either for being formally recommended on the basis of clinical trial evidence for its best-available combination of efficacy, safety, and tolerability, or for being chosen based on the clinical experience of the physician. If chemotherapy either fails to resolve the issue or produces intolerable side effects, additional, further (second-line) therapies may be substituted or added to the treatment regimen.

The term “metastasis” as described herein shall refer to the spread of malignant tumors to secondary sites, e.g., remote from an original or primary tumor. This normally involves detachment of cancer cells from a primary tumor, entering the body circulation and settling down to grow within normal tissues elsewhere in the body. Such primary tumor is understood as a tumor growing at the site of the cancer origin.

Hematopoietic diseases (leukemia, lymphomas and myeloma) are considered disseminated at time of diagnosis. However, also hematopoietic cancer can form metastatic tumors. Although rare, the metastasis of blood and lymphatic system cancers to the lung, heart, central nervous system, and other tissues has been reported. Metastatic tumor cells are understood as cells that have the ability to produce a metastasis or are already a part of a metastatic tumor.

Specifically, the primary cancer cells and/or the metastasis as referred to herein is RANK-positive, e.g., as determined by a standard immunohistochemical or a PCR-based method.

Examples of primary mesenchymal tumors are soft tissue tumors, e.g., deriving from muscle, fibrous tissue, and vascular tissue.

Among primary mesodermal and/or ectodermal tumors are melanoma and/or ameloblastoma and primitive neuro-ectodermal tumor of the lung, respectively.

Representative primary, epithelial cell cancers include amongst others breast, prostate, lung, bladder, uterine, ovarian, brain, head and neck, esophageal, pancreatic, gastric, germ cell, and colorectal cancers.

Particular important RANK-positive tumor diseases are breast cancer, pancreatic cancer, gastric cancer, esophageal cancer, renal cell carcinoma, lung carcinoma, colon/rectal/colorectal cancer, melanoma, prostate cancer, head and neck cancer, or other diseases associated with RANK-positive tumor entities.

Among the blood cancers are leukemia, lymphoma, or myeloma.

A patient suffering from leukemia can specifically benefit from the anti-RANKL treatment as described herein, because RANK signaling into leukemic cells may e.g., enhance their proliferative potential and/or alter their resistance to anti-cancer therapeutic intervention e.g., with chemotherapy and/or kinase inhibitors.

Patients treated for cancer and primary tumors often retain a minimal residual disease related to the cancer. That is, even though a patient may have by clinical measures a complete remission of the disease in response to treatment, a small fraction of the cancer cells may remain that have escaped destruction. The type and size of this residual population is an important prognostic factor for the patient's continued treatment.

In certain embodiments, the patient has minimal residual disease after the primary cancer therapy (e.g., chemotherapy, radiation therapy and/or surgery). The antagonistic agent as described herein would be particularly combined with cytoreductive therapy or other therapeutic interventions e.g., immunotherapy, to treat minimal residual disease, and/or as maintenance therapy, e.g., as a prolonged or extended therapy after cessation of another cancer treatment. In addition, the antagonistic agent would delay the re-growth or recurrence of the cancer or tumor, or recurrence of metastasis formation in metastatic disease, e.g., by at least 1 or more months.

The term “patient” as used herein shall refer to a warm-blooded mammalian, particularly a human being. In particular, the medical use as described herein, or the respective method of treatment applies to a patient in need of prophylaxis or treatment of cancer, tumor or metastatic disease. The patient may be suffering from early stage or late stage disease, or else a patient predisposed of such disease, e.g., by genetic predisposition.

The RANKL-inhibitor as described herein is specifically provided in a pharmaceutical composition. The term “pharmaceutical composition” as described herein shall refer to a composition suitable for administering to a human, i.e. a composition containing components which are pharmaceutically acceptable. Preferably, a pharmaceutical composition comprises an active compound or a salt thereof together with a carrier, diluent or pharmaceutical excipient such as buffer, or tonicity modifier.

Stable pharmaceutical compositions are contemplated which are prepared for storage. In specific embodiments, the agent having the desired degree of purity is mixed with pharmaceutically acceptable carriers, excipients or stabilizers, and provided as lyophilized formulation, aqueous solution or oil-in-water emulsion. Typically such compositions comprise a pharmaceutically acceptable carrier as known and called for by acceptable pharmaceutical practice, see e.g., Remingtons Pharmaceutical Sciences, 16^{th} edition (1980) Mack Publishing Co. Examples of such carriers include sterilized carriers such as saline, Ringers solution or dextrose solution, optionally buffered with suitable buffers to a pH within a range of 5 to 8.

The formulations to be used for in vivo administration will need to be sterile. This is readily accomplished by filtration through sterile filtration membranes or other suitable methods.

Administration of the pharmaceutical composition comprising the RANKL-inhibitor for use as described herein is specifically by the systemic route or by parenteral administration, e.g., by the intravenous, intramuscular or subcutaneous route, but also orally, intranasally, intraotically, transdermally, mucosal, topically (e.g., gels, salves, lotions, creams, etc.), intraperitoneally, intramuscularly, intrapulmonary, vaginally, parenterally, rectally or intraocularly. Exemplary formulations as used for parenteral administration include those suitable for intraveneous, intramuscular, or subcutaneous injection as, for example, a sterile solution or suspension.

In particular, the intraveneous administration is preferred, e.g., as intraveneous infusion or as a bolus injection. Denosumab is known to be administered by the subcutaneous route. In the new indication of chemoresistance treatment, the Denosumab agent would specifically be administered such that it is available in the circulation for a prolonged period of time, thus, the subcutaneous route is specifically less preferred or avoided.

The present invention includes a pharmaceutical preparation, containing as active substance the RANKL-inhibitor in a therapeutically effective amount.

The term “therapeutically effective amount”, used herein interchangeably with any of the terms “effective amount” or “sufficient amount” of the RANKL-inhibitor as described herein, is a quantity or activity sufficient to, when administered to the subject effect beneficial or desired results, including clinical results, and, as such, an effective amount or synonym thereof depends upon the context in which it is being applied. In the context of disease, therapeutically effective amounts of the RANKL-inhibitor may be used to treat, modulate, attenuate, reverse, or affect a disease or condition that benefits from a down-regulation or reduction of chemoresistance, e.g., for substantially reducing the therapeutically effective dose of a chemotherapeutic agent, such as by at least any of 10%, 20%, 30%, 40%, 50%, 60%, 70%, 80%, or 90%; or for significantly reducing the frequency of chemotherapy or the number of treatment cycles; or for preventing or treating metastatic disease. An effective amount is intended to mean that amount of a compound that is sufficient to treat, prevent or inhibit such diseases or disorder. The amount of the RANKL-inhibitor that will correspond to such an amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

Moreover, a treatment or prevention regime of a subject (a cancer patient) with a therapeutically effective amount of the RANKL-inhibitor may consist of a single administration, or alternatively comprise a series of applications. For example, the RANKL-inhibitor may be administered at least once a year, at least once a half-year or at least once a month. However, in another embodiment, the RANKL-inhibitor may be administered to the subject from about one time per week to about a daily administration for a given treatment. The length of the treatment period depends on a variety of factors, such as the severity of the disease, the age of the patient, the concentration and the activity of the antagonistic agent. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

A therapeutically effective amount of the RANKL-inhibitor such as provided to a human patient in need thereof may specifically be in the range of 0.5-1000 mg, preferably 1-400 mg, even more preferred up to 300 mg, up to 200 mg, up to 100 mg or up to 10 mg, though higher doses may be indicated e.g., for treating acute disease conditions, such as when preparing for a surgical intervention, or shortly after a surgical intervention, when starting treatment within a 1-7 days following surgery. Subcutaneous doses typically are ranging within 0.5 and 400 mg.

The term “treatment” as used herein shall always refer to treating patients for prophylactic (i.e. to prevent a disease or disease condition) or therapeutic (i.e. to treat a disease or disease condition) purposes. Treatment of a patient will typically be therapeutic in cases of cancer. However, in case of patients suffering from a primary disease, which are at risk of disease progression or at risk of developing a secondary disease condition or side reaction, e.g., which is dependent on the RANK-RANKL signalling effects, the treatment may be prophylactic. Such treatment for prophylaxis is herein also referred to as treatment or therapy, e.g., employing a therapeutically effective amount.

As described herein, the RANKL-inhibitor is administered in combination with one or more other therapeutic agents, including but not limited to standard treatment, e.g., chemotherapeutics to treat malignant disease.

The term “in combination” when used in reference to administration is understood herein as the concomitant or essentially simultaneous or sequential administration of at least two compounds, including but not limited to the two compounds, the RANKL-inhibitor and the chemotherapeutic agent. Such compounds may be administered sequentially with each other, with the term “in combination” not being limited in the sequence of administration; encompassing when a compound is administered either prior to or after administration of another compound. A compound may also be administered “in combination” with another compound when both are administered essentially at the same time or simultaneously, including e.g., when appropriate when both compounds are formulated as single dosage form. When formulated in separate dosage forms, administration may be through the same or different route of administration such as further described herein.

In a combination therapy, the RANKL-inhibitor may be administered as a mixture, or concomitantly with one or more other therapeutic regimens, e.g., either before, simultaneously (concurrently) or after chemotherapy. Concomitant administration specifically comprises administration at or almost at the same time, one after the other within a short time period of e.g., within one or two weeks.

The biological properties of the RANKL-inhibitor may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the agent to be used as a therapeutic with the appropriate half-life, effector function, apoptotic activity and IgG inhibitory activity. Any organism, preferably mammals, may be used for testing. For example, because of their genetic similarity to humans, primates, monkeys can be suitable therapeutic models, and thus may be used to test the efficacy, toxicity, pharmacokinetics, pharmacodynamics, half-life, or other property of the active agent. Tests of the substances in humans are ultimately required for approval as drugs, and these experiments are contemplated herein. Thus the active agent may be tested in animal models or in humans to determine their therapeutic efficacy, toxicity, immunogenicity, pharmacokinetics, and/or other clinical properties. Denosumab is a commercially available product with well-established biological properties, though the effect of resensitizing chemoresistant cancer cells turned out to be surprising.

The term “RANKL-inhibitor” as used herein shall refer to a RANKL specific antagonistic agent, herein also referred to as “antagonistic RANKL-inhibitor”, in particular a compound, which is a RANKL binder substantially neutralizing RANKL, and/or reducing, or inhibiting binding of RANKL to its receptor RANK, thereby antagonizing the RANK-RANKL signalling pathway, and/or substantially neutralizing RANKL and/or reducing, or inhibiting binding of RANKL to its receptor which is the leucine-rich repeat-containing G-protein-coupled receptor 4 (LGR4, also called GPR48), see Luo et al. 2016, Nat Med, 22(5):539-46.

The antagonistic function of the RANKL-inhibitor is specifically characterized by diminishing, inhibiting, or preventing a cellular response to a receptor (e.g., RANK, or LGR4) activated by an agonist (RANKL). Antagonists specifically are competitive antagonists, which can reversibly bind to the RANKL at the same binding site or interfering with the binding site (active site), as the endogenous receptor, without necessarily activating the receptor.

In addition, the RANKL-antagonist may also serve to prevent effects of RANKL reverse signaling into a tumor cell that expresses mRANKL. This comprises but is not limited to RANKL-induced secretion of immuno-inhibitory cytokines, induction of epithelial to mesenchymal transition (EMT), chemotherapy resistance, and immune escape (Schmiedel et al. 2013, J Immunol, 15; 190(2):821-31).

The RANKL-inhibitor can be any suitable binder or ligand, e.g., selected from the group consisting of small organic or inorganic molecules, carbohydrates, biological macromolecules, peptides, proteins (herein also referred to as polypeptides), peptide analogs, peptidomimetics, antibodies, including antigen-binding fragments of antibodies, nucleic acids, nucleic acid analogs, and a combination of any of the foregoing. In some embodiments, the RANKL-inhibitor is an immunotherapeutic agent. A specific example of the RANKL-inhibitor is selected from the group consisting of an antibody, a receptor or osteoprotegerin (which is inactive or rendered inactive, in order to avoid agonistic RANKL binding to its receptor RANK), receptor-fusion protein, e.g., a RANK-Fc fusion protein, a peptide, aptamer, or a small molecule.

Methods for producing and characterizing an antagonistic RANKL-inhibitor are well-known in the art. In a preferred embodiment, antagonistic binders are produced and screened for predefined properties using one or more cell-based assays. Such assays often involve monitoring the response of cells to a binder, for example cell survival, cell death, change in cellular morphology, RANKL-induced secretion of immuno-inhibitory cytokines, induction of EMT, chemotherapy resistance, and immune escape, or transcriptional activation such as cellular expression of a natural gene or reporter gene.

The production of the recombinant polypeptide RANKL-inhibitor preferably employs an expression system to produce the recombinant polypeptide, e.g., including expression constructs or vectors comprising a nucleotide sequence encoding the polypeptide.

In one embodiment, the antagonistic RANKL-inhibitor is identified through a drug discovery process, such as including a screen employing combinatorial libraries (random or semi-random) containing potential drug candidates, e.g., peptide libraries, antibody libraries, or chemical compound libraries. Screens may be performed in a high throughput manner using e.g., flow cytometry, and optionally can discriminate between active and non-active or blocked RANK/RANKL interaction. Biological screens may aim at finding novel antagonistic agents specifically targeting RANKL.

By “substantially reducing or inhibiting” the RANK-RANKL signaling, or the RANKL-LGR4 interaction, it is meant that the antagonistic RANKL-inhibitor (i) inhibits the binding of RANKL to its receptor by more than 50%, preferably more than 60%, 70%, 80%, 90% or 95%, or completely inhibits such binding; and/or (ii) functionally inhibits the RANKL-induced pathway, and in particular the signalling following RANK stimulation, e.g., activities of MAPK (Mitogen-Activated Protein Kinase) or SRC-Kinases, or NF-κB signals involved in metastasis formation. Such functional inhibition is e.g., inhibiting metastasis formation by more than about 50%, 60%, 70%, 80%, 90% or 95%, or complete inhibition.

Alternatively, the functional inhibition may be determined ex vivo, e.g., determining the migration of cancer cells via cytoskeletal rearrangements brought on by the activation of Erk1/2 and Src in a standard assay. Specifically, migration and invasion potential of tumor cells may be measured by determining the portion of cells that have passed a porous and/or extracellular-matrix mimicking barrier. Such functional inhibition is e.g., inhibiting migration and/or invasion by more than about 50%, 60%, 70%, 80%, 90% or 95%, or complete inhibition.

The functional inhibition may also be determined by measuring the downregulation of a epithelial-mesenchymal transition (EMT) gene signature, in particular metastasis-associated genes in cancer cells by targeting RANKL, e.g., by quantitative PCR-based methods, determining any of E-Cadherin, Claudin, SNAIL, or Fibronectin.

The term “antibody” as used herein shall refer to polypeptides or proteins that consist of or comprise antibody domains, which are understood as constant and/or variable domains of the heavy and/or light chains of immunoglobulins, with or without a linker sequence. The antibody as used herein has a specific antigen-binding site to bind the RANKL antigen or one or more epitopes of such antigen, specifically comprising a CDR binding site of a single variable antibody domain, such as VH, VL or VHH, or a binding site of pairs of variable antibody domains, such as a VLNH pair, an antibody comprising a VLNH domain pair and constant antibody domains, such as Fab, F(ab′), (Fab)₂, scFv, Fv, or a full length antibody.

Specific antibody formats may be used according to the invention, e.g., an antibody comprising or consisting of single variable antibody domain, such as VH, VL or VHH, or combinations of variable and/or constant antibody domains with or without a linking sequence or hinge region, including pairs of variable antibody domains, such as a VLNH pair, an antibody comprising or consisting of a VLNH domain pair and constant antibody domains, such as heavy-chain antibodies, Fab, F(ab′), (Fab)₂, scFv, Fd, Fv, or a full-length antibody, e.g., of an IgG type (e.g., an IgG1, IgG2, IgG3, or IgG4 subtype), IgA1, IgA2, IgD, IgE, or IgM antibody. The term “full length antibody” can be used to refer to any antibody molecule comprising at least most of the Fc domain and other domains commonly found in a naturally occurring antibody monomer. This phrase is used herein to emphasize that a particular antibody molecule is not an antibody fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody, e.g., with at least one other antibody, such as monoclonal antibodies or antibody fragments having different specificities.

The term “antibody” shall apply to antibodies of animal origin, including human species, such as mammalian, such as human or murine, or avian, such as hen, which term shall particularly include recombinant antibodies that are based on a sequence of animal origin, e.g., human sequences.

The term “antibody” further applies to chimeric antibodies with sequences of origin of different species, such as sequences of murine and human origin.

The term “chimeric” as used with respect to an antibody refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

The term “antibody” may further apply to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions (FR) in the variable domains. Antigen-binding sites may be wild-type or modified, e.g., by one or more amino acid substitutions, preferably modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “antibody” further applies to human antibodies.

The term “human” as used with respect to an antibody, is understood to include antibodies having variable and constant regions derived from human germline immunoglobulin sequences. The human antibody of the invention may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

The term “antibody” specifically applies to antibodies of any class or subclass. Depending on the amino acid sequence of the constant domain of their heavy chains, antibodies can be assigned to the major classes of antibodies IgA, IgD, IgE, IgG, and IgM, and several of these may be further divided into subclasses (isotypes), e.g., IgG1, IgG2, IgG3, IgG4, IgA1, and IgA2.

The term further applies to monoclonal or polyclonal antibodies, specifically a recombinant antibody, which term includes all antibodies and antibody structures that are prepared, expressed, created or isolated by recombinant means, such as antibodies originating from animals, e.g., mammalians including human, that comprises genes or sequences from different origin, e.g., murine, chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

Antibody domains may be of native structure or modified by mutagenesis or derivatisation, e.g., to modify the antigen binding properties or any other property, such as stability or functional properties, such as binding to the Fc receptors FcRn and/or Fcgamma receptor (FCGR). Polypeptide sequences are considered to be antibody domains, if comprising a beta-barrel structure consisting of at least two beta-strands of an antibody domain structure connected by a loop sequence.

It is understood that the term “antibody” also refers to derivatives of an antibody, in particular functionally active derivatives, herein also referred to as functional variants of antibodies. An antibody derivative is understood as any combination of one or more antibody domains or antibodies and/or a fusion protein, in which any domain of the antibody may be fused at any position of one or more other proteins, such as other antibodies, e.g., a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic interaction, di-sulfide bonding etc. The other substances bound to the antibody may be lipids, carbohydrates, nucleic acids, organic and inorganic molecules or any combination thereof (e.g., PEG, prodrugs or drugs). In a specific embodiment, the antibody is a derivative comprising a drug, e.g., to obtain an antibody-drug conjugate. Specifically, the antibody may be used together with a tag. Thus, the antibody may be a derivative comprising a tag, such as for analytical or diagnostic purposes, including e.g., for use as in vivo diagnostic. There is not a specific limitation with respect to the usable tag, as far as it has no or tolerable negative impact on the binding of the antibody to its target antigen. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself, e.g., radioisotope labels or fluorescent labels, or, in the case of an enzymatic label, may catalyze chemical alteration of a substrate compound or composition which is detectable.

The term derivative also includes fragments, variants, analogs or homologs of antibodies, e.g., with a specific glycosylation pattern, e.g., produced by glycoengineering, which are functional and may serve as functional variants, e.g., binding to the specific target.

The term “glycoengineered” with respect to antibody sequences shall refer to glycosylation variants having modified immunogenic properties, ADCC and/or CDC as a result of the glycoengineering. All antibodies contain carbohydrate structures at conserved positions in the heavy chain constant regions, with each isotype possessing a distinct array of N-linked carbohydrate structures, which variably affect protein assembly, secretion or functional activity. IgG1 type antibodies are glycoproteins that have a conserved N linked glycosylation site at Asn297 in each CH2 domain. The two complex bi-antennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC). Removal of N-Glycan at N297, e.g., through mutating N297, e.g., to A, or T299 typically results in aglycosylated antibodies with reduced ADCC.

Major differences in antibody glycosylation occur between cell lines, and even minor differences are seen for a given cell line grown under different culture conditions. Expression in bacterial cells typically provides for an aglycosylated antibody.

Antibodies can be devoid of an active Fc moiety, thus, either composed of antibody domains that do not have an FCGR binding site, specifically including any antibody devoid of a chain of CH2 and CH3 domains, or comprising antibody domains lacking Fc effector function, e.g., by modifications to reduce Fc effector functions, in particular to abrogate or reduce ADCC and/or CDC activity. Such modifications may be effected by mutagenesis, e.g., mutations in the FCGR binding site or by derivatives or agents to interfere with ADCC and/or CDC activity of an antibody, so to achieve reduction of Fc effector function or lack of Fc effector function, which is typically understood to refer to Fc effector function of less than 10% of the unmodified (wild-type) antibody, preferably less than 5%, as measured by ADCC and/or CDC activity.

An antibody of the present invention may or may not exhibit Fc effector function. Though the mode of action is mainly mediated by inhibiting the RANKL-RANK signaling in the tumor cell microenvironment, without Fc effector functions, Fc can recruit complement and aid elimination of the target platelet-tumor cell aggregates, from the circulation via formation of immune complexes.

Exemplary antibodies comprise an Fc fragment or at least part of an Fc fragment, such as to maintain the binding site to FcRn, thereby obtaining an antibody with substantive half-life in vivo.

A further example refers to modification of an Fc to obtain reduction of possible ADCC and/or CDC activity, e.g., by a switch of IgG1 to IgG2 subtype or by modifications to reduce binding to the Fc receptor, e.g., by E233P and/or L234V and/or L235A and/or D265G and/or A327Q and/or A330A and/or G236, deletion and/or A327G and/or A330S in a human IgG1 Fc, wherein numbering is according to Kabat [EU-Index].

Further examples refer to a modification to reduce immunogenicity, e.g., by a K.O. glycosylation site, such as N297Q, which provides for an impaired binding to lectin receptor.

An exemplary antibody is Denosumab, or a functional variant or an antigen-binding fragment thereof, e.g., incorporated in the framework of an IgG2 or any other immunoglobulin types or subtypes. For example, the Denosumab antigen-binding site or CDR sequences may be incorporated into an IgG1 antibody, with or without Fc effector function.

It is understood that the term “antibody” also refers to variants of an antibody, including antibodies with functionally active CDR variants of a parent CDR sequence, and functionally active variant antibodies of a parent antibody. For example, functional variants of Denosumab may be engineered and used as further described herein.

Specifically, an antibody variant derived from an antibody of the invention may comprise at least one or more of the CDR regions or CDR variants thereof (of the parent antibody), e.g., at least 3 CDRs of the heavy chain variable region and/or at least 3 CDRs of the light chain variable region, with at least one point mutation in at least one of the CDR or FR regions, or in the constant region of the heavy chain (HC) or light chain (LC), being functionally active, e.g., specifically binding the RANKL antigen.

The term “variant” shall particularly refer to antibodies, such as mutant antibodies or fragments of antibodies, e.g., obtained by mutagenesis methods, in particular to delete, exchange, introduce inserts into a specific antibody amino acid sequence or region or chemically derivatise an amino acid sequence, e.g., in the constant domains to engineer the antibody stability, effector function or half-life, or in the variable domains to improve antigen-binding properties, e.g., by affinity maturation techniques available in the art. Any of the known mutagenesis methods may be employed, including point mutations at desired positions, e.g., obtained by randomization techniques. In some cases positions are chosen randomly, e.g., with either any of the possible amino acids or a selection of preferred amino acids to randomize the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “functionally active variant” of an antibody means a sequence resulting from modification of this sequence (a parent antibody or a parent sequence) by insertion, deletion or substitution of one or more amino acids, or chemical derivatisation of one or more amino acid residues in the amino acid sequence, or nucleotides within the nucleotide sequence, or at either or both of the distal ends of the sequence, e.g., in a CDR sequence the N-terminal and/or C-terminal 1, 2, 3, or 4 amino acids, and/or the centric 1, 2, 3, or 4 amino acids (i.e. in the midst of the CDR sequence), and which modification does not affect, in particular impair, the activity of this sequence. In the case of a binding site having specificity to a selected target antigen, the functionally active variant of an antibody would still have the predetermined binding specificity, or substantially the same biological activity, though this could be changed, e.g., to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active CDR variant.

Preferably, an agent is used which binds to RANKL with a high affinity, in particular with a high on and/or a low off rate, or a high avidity of binding. The binding affinity is usually characterized in terms of the concentration of the agent, at which half of the binding sites are occupied, known as the dissociation constant (Kd, or KD). Usually a binder is considered a high affinity binder with a Kd<10⁻⁸ M, preferably a Kd<10⁻⁹ M, even more preferred is a Kd<10⁻¹⁰ M.

Yet, in an alternatively preferred embodiment the individual antigen binding affinities are of medium affinity, e.g., with a Kd of less than 10⁻⁶ M and up to 10⁻⁸ M, e.g., when binding to at least two antigens.

The term “substantially the same biological activity” as used herein refers to the activity as indicated by substantially the same activity being at least 20%, at least 50%, at least 75%, at least 90%, e.g., at least 100%, or at least 125%, or at least 150%, or at least 175%, or e.g., up to 200% of the activity as determined for the comparable or parent antibody.

In a preferred embodiment the functionally active variant of a parent antibody

a) is a biologically active fragment of the antibody, the fragment comprising at least 50% of the sequence of the molecule, preferably at least 60%, at least 70%, at least 80%, at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody according to the invention is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally occurring allelic variants, as well as mutants or any other non-naturally occurring variants. As is known in the art, an allelic variant is an alternate form of a (poly)peptide that is characterized as having a substitution, deletion, or addition of one or more amino acids that does essentially not alter the biological function of the polypeptide.

Functionally active variants may be obtained by sequence alterations in the polypeptide or the nucleotide sequence, e.g., by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

The term “antigen” as used herein interchangeably with the terms “target” or “target antigen” shall refer to a whole target molecule or a fragment of such molecule recognized by an agent specifically recognizing the antigen, or capable of specifically binding the target, such as an antibody which recognizes the antigen through binding by the antibody binding site. Specifically, substructures of an antigen, e.g., a polypeptide or carbohydrate structure, generally referred to as “epitopes”, e.g., B-cell epitopes or T-cell epitope, which are immunologically relevant, may be recognized by such binding site. The term “epitope” as used herein shall in particular refer to a molecular structure which may completely make up a specific binding partner or be part of a specific binding partner to a binding site of an agent as described herein. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding the polypeptide to form a tertiary structure and the amino acids are not necessarily adjacent to one another in the linear sequence.

The target of the RANKL-inhibitor as described herein specifically is human RANKL, in particular any one or more of platelet bound RANKL pRANKL, soluble sRANKL, and/or mRANKL. An exemplary RANKL is human RANKL (GenBank: AAB86811.1) characterized by the RANKL amino acid sequence identified as SEQ ID 3, or any fragment or derivative thereof incorporating an immunorelevant epitope of RANKL.

The term “RANKL” includes any variants, isoforms and species homologs of human RANKL which are naturally expressed by cells and which are bound to the surface of cells, e.g., of human blood platelets, tumor cells, or metastasis cells, or which are present as soluble RANKL in the circulation, as determined in a sample of peripheral blood.

Preferred epitopes of RANKL are incorporated in the extracellular portion of the RANKL antigen, in particular the extracellular part of the pRANKL or the extracellular part of the transmembrane RANKL, e.g., an epitope which is accessible on the surface of the platelets or cells.

The antagonistic RANKL-inhibitor as described herein may be binding to an epitope of RANKL, which leads to substantial inhibition of the RANKL binding to its receptor, e.g., RANK, thereby inhibiting the signalling pathway, or LGR4. Since RANKL promotes survival and induces migration of various cancer cells that express RANK, the antagonistic RANKL-inhibitor as described herein would interfere with the proliferation and metastasis of cancer cells by preventing or reducing premetastatic migration and aggregation of cancer or tumor cells.

The human pRANKL is specifically understood as RANKL originating from human blood platelets (also referred to as thrombocytes), e.g., an antigen expressed on the surface of a human blood platelet, preferably by an activated platelet, which can be targeted with an antagonist that binds thereto. The platelet can also interact with a (RANKL negative) cancer cell to transform such cancer cell into a premetastatic lesion, which itself is capable of expressing RANKL. Thus, the RANKL-inhibitor may target the sRANKL and as well as targeting pRANKL and the cancer cell expressing RANKL. mRANKL may be expressed by tissue, or a cancer cell, and further interacting with a platelet and/or another cancer cell.

Overexpression of RANK or RANKL is herein understood as an increased level of producing the RANK or RANKL by a cell, e.g., thereby obtaining an elevated detectable level in a cell culture or biological sample.

The term RANK or RANKL “overexpression” as used herein shall refer to samples or cells expressing a higher amount of any of the RANK or RANKL, specifically a significantly higher amount, as compared to a reference value, which may be zero or higher, e.g., higher than a threshold or cut-off value, or higher than a reference value derived from a comparable sample. Overexpression may as well be determined by comparison to standards, including internal or external standards.

As used herein, the terms “high”, “elevated”, or “increased” with respect to the level of RANK or RANKL refer to increased amounts or a gain of function (such as the activity) of a gene product/protein compared to the wild type.

The term “significantly higher” or “significant” with respect to the overexpression of a biomarker as used herein shall refer to at least a two-fold higher amount of the standard deviation, preferably at least a three-fold difference. With respect to a specific reference value, such as derived from a standard, training data or threshold, a significant increased amount is understood to refer to an at least 1.5 fold higher amount, preferably at least 2 or 3 fold difference.

Several methods of detecting elevated levels of RANK or RANKL are known in the art. In some embodiments, overexpression is detected by detecting elevated levels of RANK or RANKL mRNA and/or protein. Levels of mRNA and/or protein can be detected using standard techniques.

In some embodiments, overexpression in a biological sample is detected in a gene expression assay. In some embodiments, the gene expression assay comprises a hybridization assay, nucleic acid amplification, a quantitative PCR (qPCR) analysis, an RNA-Sequence, or a Northern blot analysis. In some embodiments, the gene expression assay comprises a microarray analysis.

In some embodiments, overexpression in the biological sample is detected in a protein expression assay. In some embodiments, the protein expression assay comprises a Western Blot, ELISA, or other antibody-based protein expression assay. In some embodiments, overexpression in the biological sample is detected by flow cytometry, FACS (fluorescent activated cell sorting), immunohistochemistry or RNA in situ hybridization.

According to a certain aspect, increased activity can be caused by increased mRNA and/or increased protein levels. Increased mRNA levels can be caused by gene amplification and/or increased transcription, for example. Alternatively, in some embodiments, increased activity levels can be caused by a gain of function mutation resulting from a point mutation (e.g., a substitution, a missense mutation, or a nonsense mutation), an insertion, and/or a deletion, or a rearrangement in the polypeptide, or the nucleic acid sequence encoding a polypeptide, or a nucleic acid controlling the expression of a polypeptide. In some embodiments, overexpression is associated with a mutation or mutations in the gene and/or in a gene that encodes a regulator of transcription and/or translation.

The term “specific” with regard to the RANKL-specific inhibitor or antibody as described herein shall refer to a binding reaction which is determinative of the cognate ligand of interest (RANKL) in a heterogeneous population of molecules. Thus, under designated conditions, e.g., immunoassay conditions, the agent that specifically binds to its particular target does not bind in a significant amount to other molecules present in a sample.

A specific binding site or a specific agent is typically recognizing the target only, and not cross-reactive with other targets. Still, the specific binding site may specifically bind to one or more epitopes, isoforms or variants of the target, or be cross-reactive to other related target antigens, e.g., homologs or analogs.

The specific binding means that binding is selective in terms of target identity, high, medium or low binding affinity or avidity, as selected. Selective binding is usually achieved if the binding constant or binding dynamics is at least 10 fold different, preferably the difference is at least 100 fold, and more preferred a least 1000 fold.

Therefore, the invention provides for a new method of treatment, wherein chemotherapy is improved by the combination with a RANKL-inhibitor, such as Denosumab. It has been surprisingly proven that chemoresistance could be effectively treated by any such combination.

According to a specific example, it has been shown in an ex vivo cancer cell assay that the amount of viable AML cells was reduced by applying doxorubicin. This effect was neutralized in the presence of sRANKL. The amount of apoptotic and/or necrotic cells was higher upon application of doxorubicin. Again this effect was undermined by sRANKL. These data indicate that the inhibition and neutralization of RANKL by a RANKL-inhibitor, such as Denosumab, can improve the response of tumor cells to chemotherapy, i.e. overcome chemoresistance.

Further, it has been proven that the inhibition of RANKL by Denosumab resensitizes breast tumor cells to chemotherapeutic treatment. In an in vitro assay paclitaxel was applied to breast cancer cells and the percentage of living and dead cells determined. It was proven that RANKL protects malignant cells from the effect of chemotherapy, and inhibition of RANKL by Denosumab restored sensitivity to treatment.

The foregoing description will be more fully understood with reference to the following examples. Such examples are, however, merely representative of methods of practicing one or more embodiments of the present invention and should not be read as limiting the scope of invention.

### EXAMPLES

### Example 1: RANKL Mediates Resistance of Malignant Cells to Chemotherapeutic Treatment

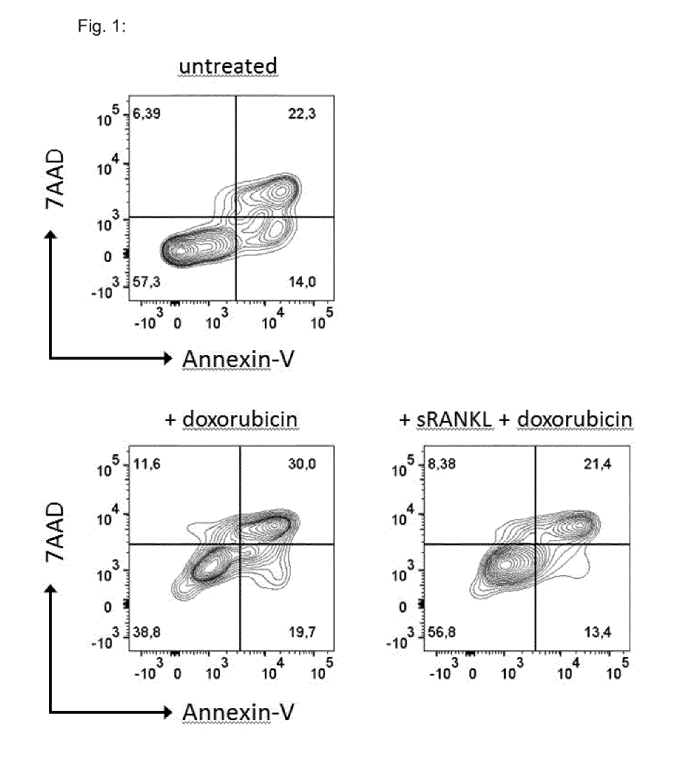

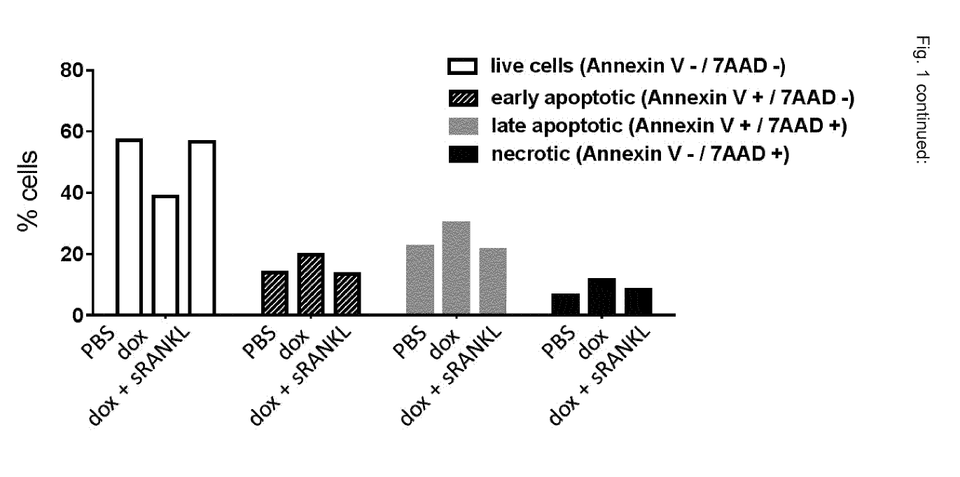

RANK expressing PBMC of a patient suffering from AML (French-American-British (FAB) classification: M4; 94% peripheral blood blasts) were subjected to a model of chemotherapyresistance (FIG. 1). To this end, patient blood was obtained at time of diagnosis prior to treatment and PBMC were isolated by density gradient centrifugation. Upon application of the in this disease commonly used first line therapeutic agent doxorubicin (1 μM, cell pharm), the percentage of early apoptotic and dead cells was greatly enhanced compared to the control group as observed by flow cytometry (PE Annexin V Apoptosis Detection Kit I, BD Pharmingen) after 48 h of treatment. The effect of chemotherapeutic treatment was markedly reduced when sRANKL (500 ng/ml, recombinant human soluble RANKL, Immunotools, Germany) was added to this regimen, which mimics the contribution of local and systemic RANKL present in a living being. The experiment demonstrates that the presence of RANKL decreases chemosensitivity and thus renders malignant cells chemoresistant. Since AML cells of the FAB type M4 resemble monocyte-like cells, this experiment is in line with studies in healthy monocytes showing that sRANKL (Seshasayee et al. 2004, Jour Biol Chem, 279(29):30202-9) acts as a pro-survival factor, likely due to upregulation of anti-apoptotic BCL-2 proteins.

### Example 2: Blockade of RANKL by Denosumab Overcomes Resistance to Chemotherapeutic Treatment

Since chemotherapy resistance is a major obstacle for the success of cancer therapy, there is an urgent need to develop new therapies to overcome chemoresistance of tumors, i.e. to increase the sensitivity of tumors to antineoplastic or chemotherapeutic drugs. In addition, reduction of the dose of chemotherapeutic drugs necessary to achieve sufficient antitumor activity would serve to maintain efficacy while at the same time reducing side effects.

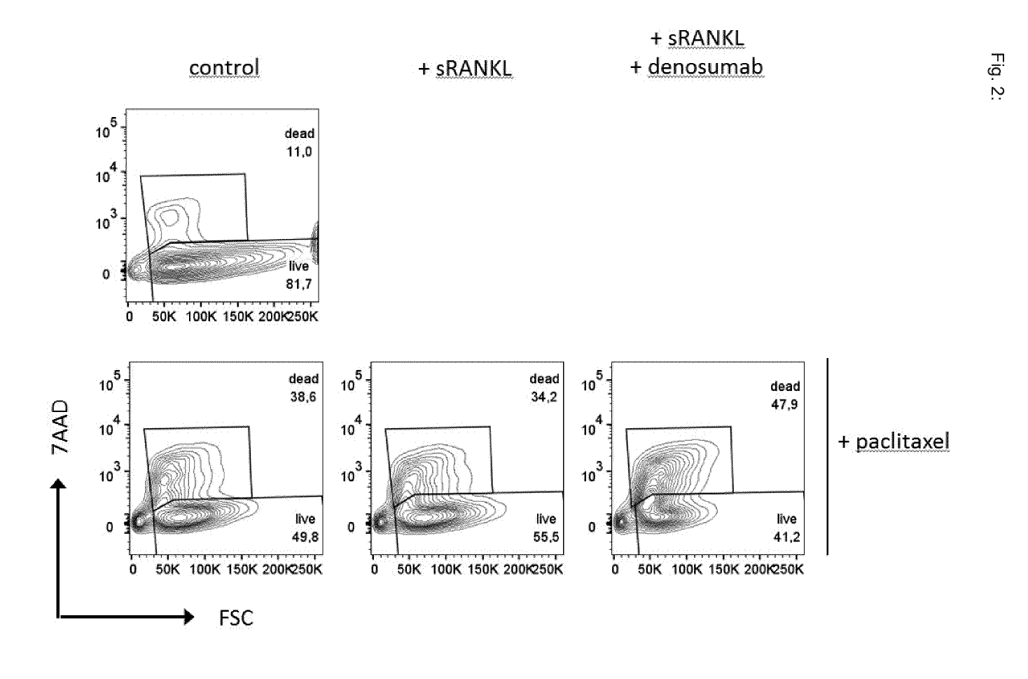

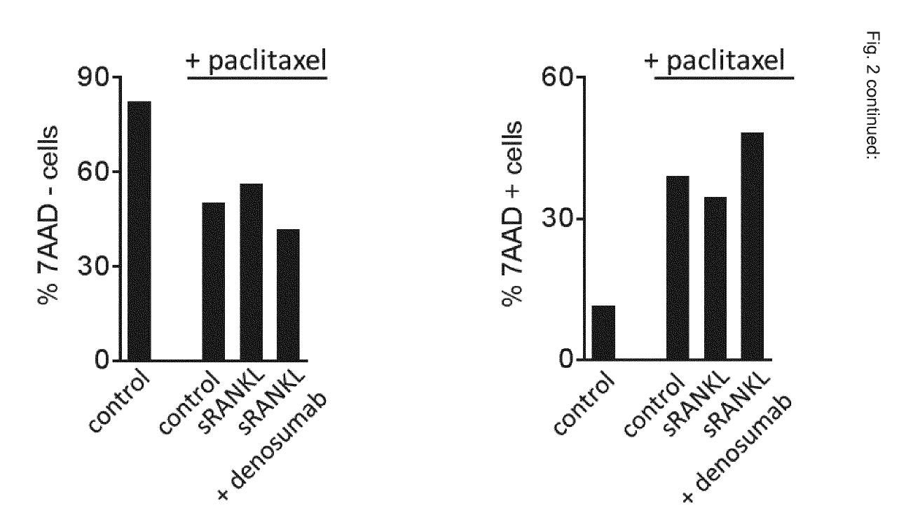

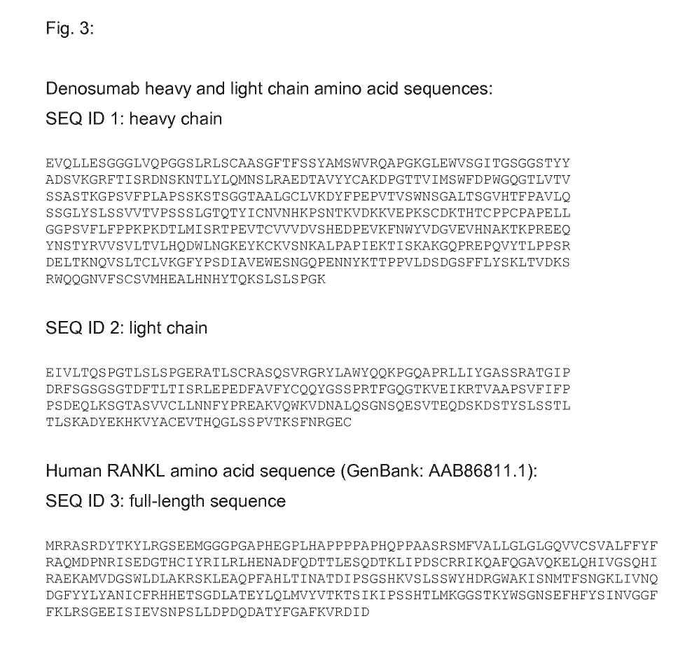

To this end, it was evaluated if RANKL-induced chemoresistance could be overcome by application of a RANKL-inhibitor in the form of the approved RANKL antibody Denosumab. Therefore, MCF10A breast cancer cells were cultured for 48 h in the presence or absence of sRANKL (100 ng/ml, Immunotools) and Denosumab (20 μg/ml, Amgen) as indicated. After 24 h of culture, paclitaxel (10 nM, Taxomedac) representing a first line breast cancer therapeutic agent, was added where indicated. Survival and cell death were analyzed after 48 h by flow cytometry staining with 7AAD (BD Pharmingen) and are displayed as contour plots (FIG. 2, upper panel) and percent of analyzed cells (lower panel). Similar to example 1 showing RANKL-induced chemoresistance in blood-borne cancers like AML, also in this breast cancer model sRANKL protects the malignant cells from the effects of chemotherapeutic treatment. This finding is in line with Schramek et al. (Nature 2010 468(7320):98-102) who found that sRANKL protects the SKBR3 breast cancer cell line from apoptosis induced by doxorubicin. However, the experiment shown in FIG. 2 further demonstrates that RANKL neutralization by a RANKL-inhibitor, in this case by Denosumab, restores and even increases the effects of chemotherapeutic treatment and thus is an effective and feasible strategy to restore chemosensitivity to overcome chemoresistance in cancer.

### Example 3: Neutralization of pRANKL Enhances Susceptibility to Chemotherapeutic Treatment

To assess the role of pRANKL in the context of systemic treatment, the viability of ovarian and breast cancer cells was analyzed. Cells were incubated with human platelets in the presence or absence of Denosumab to neutralize pRANKL which mimics the situation in cancer patients. Presence of platelets enhanced the viability/cellular activity of tumor cells already without chemotherapeutic treatment, and the same was observed upon chemotherapeutic treatment.

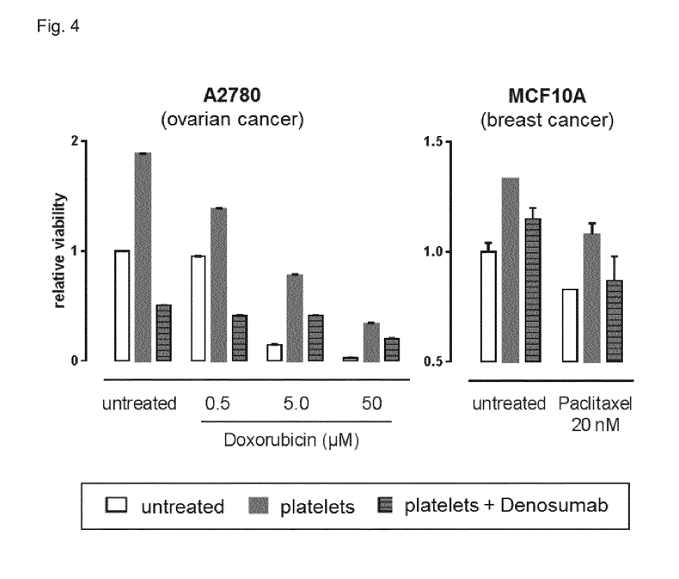

FIG. 4 shows that the protective effect of platelets was substantially reduced when platelet-derived RANKL had been neutralized by presence of Denosumab in the cultures. These data indicate that targeting pRANKL re-sensitizes cancer cells for chemotherapeutic treatment. It could be shown that neutralisation of RANKL by Denosumab increases tumor cell viability upon chemotherapeutic treatment, i.e. chemosensitivity.

The examples show a profound treatment effect of RANKL-inhibitors such as Denosumab in an AML model and models of ovarian and breast cancer, proving the concept of chemosensitizing in the respective cancer patients. The data can be confirmed in respective clinical models.

1. A RANKL-inhibitor for use in the treatment of a patient suffering from chemotherapy resistant cancer, which patient has a diagnosis of cancer that is unresponsive to treatment with a first chemotherapeutic agent, wherein said cancer is not a solid tumor or metastasis in the bone, and said patient is administered a second chemotherapeutic agent in combination with said RANKL-inhibitor.

2. The RANKL-inhibitor for use according to claim 1, wherein said patient has failed treatment with said first chemotherapeutic agent.

3. The RANKL-inhibitor for use according to claim 1 or 2, wherein said cancer is unresponsive to treatment with said first chemotherapeutic agent as determined in an ex vivo assay, and/or which is otherwise known to be chemotherapy resistant.

4. The RANKL-inhibitor for use according to any one of claims 1 to 3, wherein said patient has suffered disease progression after said treatment with said first chemotherapeutic agent including a cytotoxic chemotherapy regime, preferably a minimum of two courses of one prior cytotoxic chemotherapy regime for said cancer.

5. The RANKL-inhibitor for use according to any one of claims 1 to 4, wherein said first chemotherapeutic agent is a cytostatic, cytotoxic or any other anti-neoplastic compound used for treating a cancer disease.

6. The RANKL-inhibitor for use according to any one of claims 1 to 5, wherein said second chemotherapeutic agent is administered at a substantially reduced dose as compared to standard chemotherapy without combination with said RANKL-inhibitor.

7. The RANKL-inhibitor for use according to any one of claims 1 to 6, wherein said first chemotherapeutic agent is the same as said second chemotherapeutic agent.

8. The RANKL-inhibitor for use according to any one of claims 1 to 7, wherein said cancer is a solid tumor selected from the group consisting of epithelial tumors, mesenchymal tumors, tumors of endodermal, mesodermal and/or ectodermal origin, or a blood-borne cancer.

9. The RANKL-inhibitor for use according to any one of claims 1 to 8, wherein said patient is a carcinoma patient suffering from any of breast cancer, pancreatic cancer, gastric cancer, esophageal cancer, renal cell carcinoma, lung carcinoma, colon cancer, rectal cancer, colorectal cancer, melanoma, ovary cancer, liver cancer, kidney cancer, intestine cancer, prostate cancer, or head and neck cancer.

10. The RANKL-inhibitor for use according to any one of claims 1 to 9, wherein said patient is a blood cancer patient suffering from leukemia or lymphoma.

11. The RANKL-inhibitor for use according to any one of claims 1 to 10, wherein the RANKL-inhibitor is any of a human or humanized antibody, an antigen-binding fragment thereof, a RANKL receptor-Fc fusion protein, a molecule comprising the extracellular domain of a RANKL receptor, or a small molecule inhibitor.

12. The RANKL-inhibitor for use according to any one of claims 1 to 11, wherein the RANKL-inhibitor is administered to the patient in a therapeutically effective amount by systemic administration, preferably by intravenous infusion or bolus injection.

13. The RANKL-inhibitor for use according to any one of claims 1 to 12, wherein the respective amounts of the RANKL-inhibitor and said second chemotherapeutic agent are effective to increase the cancer sensitivity and/or treat cancer cell resistance to the chemotherapeutic agent.

14. A RANKL-inhibitor for use in the treatment of a cancer patient in combination with an anti-cancer treatment, wherein the anti-cancer treatment is administered at a substantially reduced dose as compared to standard treatment without combination with said RANKL-inhibitor.

15. The RANKL-inhibitor for use according to claim 14, wherein said cancer is not a solid tumor or metastasis in the bone.

16. A Method for screening patients suffering from a cancer and who might benefit from a therapy for sensitizing the cancer cells to chemotherapy, said method comprising the step of measuring the level of expression and/or activity of RANK or RANKL in a biological sample of said patient, wherein patients who show an expression or an overexpression of RANK or RANKL are selected as a candidate for said sensitizing therapy.

17. The method of claim 16, wherein said cancer is not a solid tumor or metastasis in the bone.

18. Method according to claim 16 or 17, wherein said biological sample is selected from the group consisting of tissue samples obtained from a biopsy procedure or from a surgical procedure to remove a tumor mass, blood, which may comprise tumor derived material such as tumor cells or tumor relapsed proteins and/or nucleic acids.

19. Method according to any one of claims 16 to 18, wherein said method further comprises comparing the level of expression of RANK or RANKL to a threshold value, and wherein said threshold value is the mean level of expression of a population of patients who are healthy or who recovered from a cancer.

20. The RANKL-inhibitor for use according to any one of claims 1 to 14, wherein said patient has been identified as candidate for said treatment according to the method of any one of claims 16 to 19.
